(19) United States
(12) Patent Application Publication
(10) Pub. No.: US 2025/0248854 A1
(43) Pub. Date: Aug. 7, 2025

(54) METHOD AND SYSTEM FOR INTEGRATING AN ADDITIVE TO A SUBSTRATE

(71) Applicant: Biodaptive Advanced Materials, LLC, Ronkonkoma, NY (US)

(72) Inventors: Daniel Rivest, Charlotte, VT (US); Patrick John Lamb, New Port Richey, FL (US); Matthew Robert Henry, State College, PA (US); Michael R. Bielski, Manorville, NY (US)

(21) Appl. No.: 18/854,378

(22) PCT Filed: Apr. 7, 2023
(86) PCT No.: PCT/US2023/017892

§ 371 Date: Oct. 4, 2024

Related U.S. Application Data
(60) Provisional application No. 63/328,481, filed on Apr. 7, 2022.

(51) Int. Cl. A61F 13/00

(52) U.S. Cl. CPC A61F 13/00991; A61F 13/00063

(57) ABSTRACT

A system and method for integrating an additive into a substrate to change the characteristics of the substrate includes a roller to impregnate or integrate the additive into the substrate and a heating element to apply heat and improve adhesion of the additive to the substrate.

### CROSS-REFERENCE TO RELATED APPLICATION

The present application claims benefit of and priority to U.S. Provisional Patent Application No. 63/328,481 filed Apr. 7, 2022 entitled METHOD AND SYSTEM FOR INTEGRATING AN ADDITIVE TO A SUBSTRATE, the entire content of which is incorporated by reference herein.

### FIELD OF THE DISCLOSURE

The present invention relates to a method and system for integrating an additive to a substrate which may be included in a bandage suitable for use in wound care.

### RELATED ART

Bandages including absorbent material, which may be embodied in a porous substrate, and are typically provided to cover wounds while they heal. In embodiments, bandages may include other materials that may not be porous, such as the skin contacting layer. Bandages may also be used as a preventative measure to prevent formation of sores or wounds. Bandages may be used in conjunction with medications or other substances to aid in treatment, however, such substances are typically applied directly to the user's skin and covered by the bandage. Such an application may affect how the bandage adheres to the skin and may provide unequal application of the substance over the wound. In addition, bandages, may be or may become contaminated with microorganisms which may raise the risk of contamination of the wound that is being covered by the bandage. Further, excess moisture in a wound may delay wound healing which may result in excessive scarring, maceration and inflammation at the wound.

Accordingly, it would be beneficial to provide a substrate for use in a bandage that avoids these and other problems.

### SUMMARY

It is an object of the present disclosure to provide a method and system for integrating an additive into a substrate which may be a part of a bandage or wound dressing used to treat a wound, wherein the additive may aid in healing or protection of the wound.

A system of integrating an additive into a substrate includes a distribution element configured to hold a first amount of the additive; an applicator operably connected to the distribution element to receive the additive and apply the additive to the substrate; and a conveyor operable to advance the substrate under the applicator, wherein the additive is applied to the substrate by the applicator such that the additive is integrated into the substrate.

In embodiments, the additive is a powder.

In embodiments, the applicator is a roller positioned to contact the substrate such that the roller presses the additive into the substrate.

In embodiments, the additive includes an adhesive such that the additive remains integrated into the substrate.

In embodiments, the substrate includes an adhesive configured to retain the additive in the substrate.

In embodiments, the additive is electrostatically charged.

In embodiments, the system includes: a first electrode positioned above the substrate; a second electrode positioned below the substrate, wherein an AC electrical field is established across the substrate between the first electrode and the second electrode to drive the additive through the substrate.

In embodiments, the first electrode is provided on the roller.

In embodiments, the first electrode is the roller.

In embodiments, a distribution of the additive through the substrate depends on a voltage provided across the AC electrical field between the first electrode and the second electrode.

In embodiments, the additive is one of a liquid and a semi-liquid.

In embodiments, the applicator is positioned above the substrate with a gap provided between the applicator and the substrate to allow a layer of the additive to be provided on the substrate.

In embodiments, a heating element configured to receive the substrate after the additive is integrated into the substrate to heat the substrate and the additive, wherein the additive at least partially melts to improve adhesion to the substrate.

In embodiments, the heating element heats the additive to activate one or more characteristics thereof.

A method of integrating an additive to a substrate includes: providing the additive to a distribution element; advancing the substrate under an applicator operably connected to the distribution element; and applying the additive to the substrate as the substrate passes under the applicator such that the additive is integrated into the substrate. In embodiments, the additive is a powder.

In embodiments, the method may include charging the powdered additive electrostatically.

In embodiments, the step of applying the additive includes applying an AC voltage across a depth of the substrate, wherein varying the AC voltage varies a distribution of the additive through the depth of the substrate.

In embodiments, the method includes, after the step of applying the additive, applying heat to the substrate and the additive to at least partially melt the additive and improve adhesion to the substrate.

In embodiments, the step of applying heat to the substrate and the additive activates a characteristic of the additive.

In embodiments, the additive is one of a liquid and a semi-liquid.

In embodiments, the step of applying the additive includes applying a layer of the additive on the substrate.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 illustrates a schematic representation of a system for integrating an additive to a substrate in accordance with an exemplary embodiment of the present disclosure;

FIG. 1A illustrates a more detailed view of the contact between a roller of the system and the substrate to which the additive is integrated in accordance with an exemplary embodiment of the present disclosure;

FIG. 1B illustrates a more detailed view of a heating portion of the system of FIG. 1 in accordance with an exemplary embodiment of the present disclosure;

FIG. 2 illustrates an exemplary flow chart illustrating exemplary steps used to integrate an additive into a substrate in accordance with an exemplary embodiment of the present disclosure; and

FIGS. 3A-3E illustrate cross-sectional views of the substrate illustrating various distributions of the additive through the substrate.

### DETAILED DESCRIPTION OF THE EXEMPLARY EMBODIMENTS

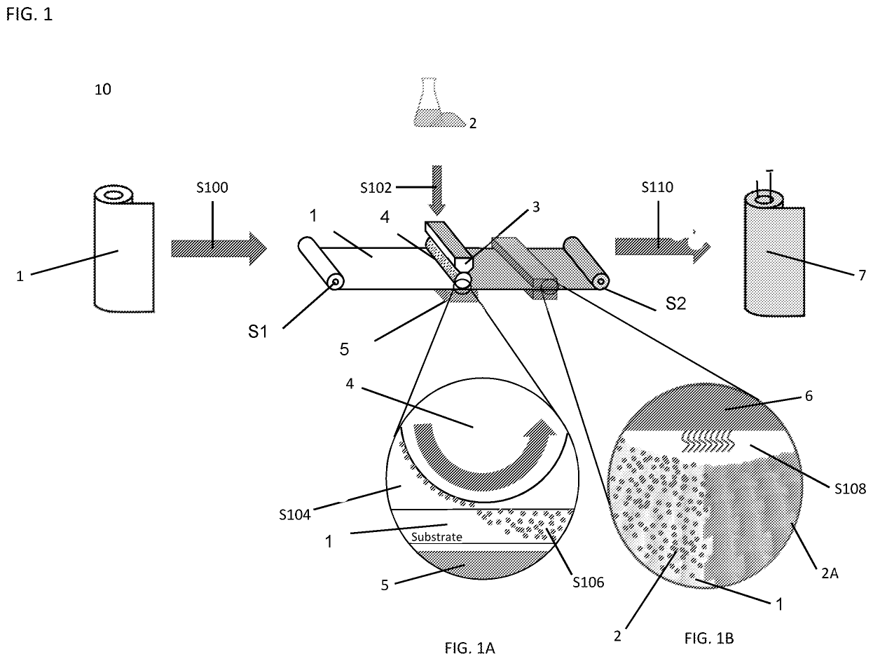

A system 10 in accordance with an embodiment of the present disclosure is illustrated in FIG. 1. In embodiments, the system 10 may be used to integrate an additive 2 into a substrate 1. In embodiment, the system 10 may include a roller 4, or other applicator, positioned such that the substrate 1 passes under it. In embodiments, an additive 2 is integrated into or impregnated in the substrate 1 via the system 10. In embodiments, the substrate 1 may be a porous material and may be, or may be a part of, a bandage or wound dressing suitable for use in wound care. In embodiments, the porous substrate 1 may be any material used for wound care. In embodiments, the porous substrate 1 may be made of one or more absorbent materials, such as medical grade polyurethane foam, super absorbent fiber, super absorbent polymer, hydrocolloid, carboxymethyl cellulose, alginate, spunbound polypropylene, or meltblown polypropylene, to name a few.

In embodiments, the substrate 1 may be a non-porous material and may be part of a bandage or wound dressing suitable for use in wound care. In embodiments, the non-porous substrate 1 may be any material used for wound care including the skin contacting layer of a bandage or dressing. In embodiments, the non-porous substrate 1 may be made of one or more materials, such as pressure sensitive adhesives, polyurethane film, or silicone gel to name a few.

In embodiments, the additive 2 may be provided in powder form. In embodiments, the additive 2 may be applied to the substrate via the roller 4, or other applicator. In embodiments, the powder-based additive 2 modifies the substrate 1 to provide characteristics or properties that are beneficial to either the manufacturing process of a medical dressing and/or that provide properties that improve the overall performance and/or safety of the substrate 1 when used as a component within a medical dressing. In embodiments, such characteristics or properties that are modified by powder-based additive 2 may include surface tension, wettability, absorption capacity, adhesive properties, protein adsorption, antibacterial activity, antifungal activity, and antiviral activity, to name a few. In embodiments, the powder-based additive 2 may include one or more additives, such as any of the any of the additives discussed above and antimicrobial agents, emollients, wound cleansers, extracellular matrix (ECM) components (e.g. collagen), thermoplastic polymers, thermosetting polymers, and any other additive that may prove beneficial to wound healing therapy.

In embodiments, an adhesive, binder or binder system may be added to either the substrate 1 and/or the powder-based additive 2 prior to the additive being applied to the substrate. In embodiments, the adhesive, binder or binder system may be part of, that is, mixed in with the additive 2.

In embodiments, the substrate 1 may be pre-treated with an adhesive or binder. In embodiments, adding an adhesive, binder or binder system to the additive 2 or substrate 1 improves attachment/anchoring of the additive to the substrate after it has been integrated or impregnated into the material thereof. In embodiments, this prevents the additive from leaching or migrating out of the dressing and into the wound during use.

While the system 10 is preferably used to provide an additive 2 to a substrate 1 used in a bandage or other wound dressing, the system 10 may be used to integrate an additive into substrates for use in other applications.

In embodiments, the additive 2 may be a liquid or semi-liquid (i.e. viscous liquids/gels). In embodiments, where the additive 2 is a liquid or semi-liquid, the electrostatic powder impregnation process discussed below to apply and distribute the additive to the substrate would generally not be beneficial. In embodiments, where a liquid or semi-liquid additive 2 is used, a stamping approach may be used to apply the additive to the substrate. In embodiments, where a liquid or semi-liquid additive is used, it may be applied as a thin film of liquid/gel that absorbs into the bulk material of the substrate or adsorbs to the surface of the material of the substrate.

In embodiments, the powder additive 2 may be provided to a distribution element 3, such as a hopper or other suitable structure such that the additive is substantially evenly applied over a length of the roller 4 or other applicator. In embodiments, the powder additive 2 drops from the distribution element 3 onto the roller 4 and as the roller makes contact with the substrate 1, the additive is pressed into the substrate 2 as can generally be seen in FIG. 1A. In embodiments, where a liquid or semi-liquid additive 2 is used, a small gap may be provided between the roller 4 and the substrate 1 to allow for stamping or application of a thin film of liquid/gel to the substrate. In embodiments, no gap may be provided. In embodiments, a size of the gap may be adjustable.

In embodiments, an AC electric field may be applied across either side of the porous substrate 1, perpendicular to the direction of travel of the substrate through the system 10. In embodiments, the substrate 1 may travel on a conveyor system under the roller 4. In embodiments, the conveyor system may include a first spindle S1 and a second spindle S2 positioned on opposite sides of the of the system 10. The spindle S1 advances the substrate 1 into the system 10 and the spindle S2 draws the substrate with the additive 2 integrated or impregnated therein past the roller 4 and out of the system 100. In embodiments, the AC electric field may further drive the powder-based additive 2 across the entire thickness of the porous substrate 1. In embodiments, the AC electric field may be used to create a uniform distribution of powder-based additive 2 across the entire thickness of porous substrate 1 and/or to create a controlled concentration gradient of powder-based additive 2 across the thickness of porous substrate 1. In embodiments, particles of the additive 2 may be charged. In embodiments, the dry powder additives 2 may be provided in a high voltage alternating electric field to charge the particles of the additive. In embodiments, the charge may be formed on the powder additive 2 by friction (e.g. based on the tribioelectric effect, tribioelectric charging or tribiocharging). In embodiments, tribiocharging of the particles may take place when transporting a powder from a reservoir to spray gun, coater or other applicator, such as the roller 4.

In embodiments, the electrostatic charge on the powder additive 2 may be used to: (1) attract the powder-based additive 2 to the substrate 1 (as long as the substrate exhibits the opposite charge); (2) prevent agglomeration of the powder additive within or on the substrate since the particles of the additive 2 have the same charge, and thus, repel one another; and (3) allows for the use of a controllable high voltage alternating electric field provided by electrodes placed on either side of the substrate 1 to drive the charged particles into the material of the substrate to various depths for uniformity. In embodiments, where a liquid or gel additive 2 is used, it will generally not be charged.

In embodiments, the AC electric field may be applied across substrate 1 utilizing two or more electrodes arranged on either side of the porous substrate. In embodiments, the roller 4 may serve as an electrode, or may include an electrode, while another electrode 5 may be positioned on the other side of substrate 1. In embodiments, the intensity of the AC electric field may be adjusted to control the distance powder-based additive 2 is distributed across the thickness of substrate 1. Additionally, in embodiments, the AC electric field may be adjusted to control the concentration gradient of powder-based additive 2 across the thickness of the porous substrate 1. In embodiments, the voltage of the AC electric field and the duration of time that the field is applied to the substrate 1 will dictate the distribution of the powder-based additive 2 throughout the substrate. In embodiments, when the substrate 1 passes through a high voltage electric field, the electric field will cause the charged powder-based additive 2 to migrate from the top surface of a porous substrate where it is originally applied, down toward the bottom surface of the substrate. In embodiments, the stronger (higher) the voltage, the more the powder-based additive is drawn into the porous substrate 1 and the further it penetrates. In embodiments, the longer the electric field is applied, the further the powder-based additive will penetrate into the porous substrate. In embodiments, the voltage of the AC electric field may be between 0.1 to 50 kV, however, other voltages may be used. In embodiments, voltage and exposure time to the electric field may vary based on: (1) the porosity of the substrate material, (2) grain size of the powder additive, (3) electrostatic charge of the powder additive, and (4) the desired depth of powder additive penetration into the material. In embodiments, substrate porosity, small powder additive grain size, high electrostatic charge retention on the powder additive, and a shallow desired penetration depth are all properties that will favor lower AC electric field voltages and/or exposure times. In embodiments, where the substrate 1 is a non-porous material, the particles of additive 2 will remain generally on the surface of the substrate.

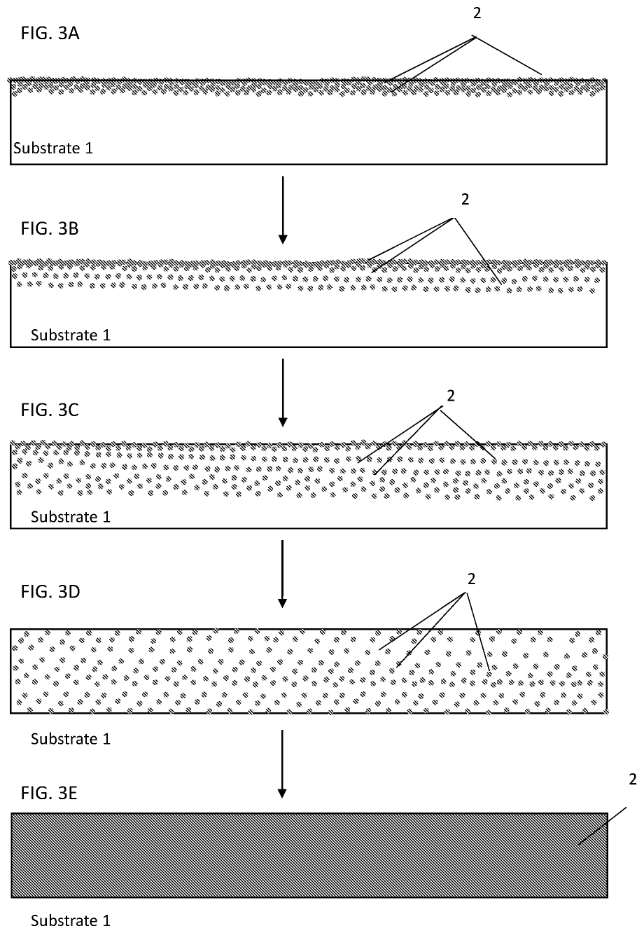

In embodiments, FIGS. 3A-3E illustrate penetration of the particles of the additive 2 into the porous substrate 1. In FIG. 3A, the particles of the additive 2 are applied to a surface of the substrate 1. In FIG. 3B, the particles begin to permeate down into the substrate 1 and continue to penetrate downward in FIGS. 3C-3D. In FIG. 3E, the particles of additive are fully integrated into the substrate 1.

In embodiments, some additives (e.g. fine metallic powders such as silver/nanosilver) may not retain an electric charge since they are conductive such that electrostatic powder integrated or impregnated into the substrate may be difficult or ineffective. In embodiments, one alternative approach is to compound such a conductive additive into a polymer carrier and then pelletize the polymer for application to the substrate 1. In embodiments, the pellets of this polymer may be ground to a fine powder which may then be applied to the substrate 1 as a powder using electrostatic powder impregnation as discussed herein. In embodiments, the polymer powder carrier may retain an electrostatic charge and makes impregnation into/onto a porous substrate using an AC electric field as discussed below possible. In embodiments, where a low melt point polymer, such as PE, is used, applying some heat may melt the polymer to the surface of the substrate and thus improve adhesion of the additive 2 to the substrate.

In embodiments, the powder-based additive 2 is adhered to the substrate 1 by either electrostatic forces or through the aid of an adhesive or binder system added to substrate 1 or powder-based additive 2 prior to the additive being integrated into the substrate. In embodiments, an electrostatic charge may be provided on the powder additive 2 using tubular electrodes arranged parallel to the substrate as it travels. In embodiments, the roller 4 or other application may be made of a conductive material and deposits the additive on the substrate. In embodiments, a single pole output is used to connect the electrodes to a high voltage electrostatic generator and the roller or applicator is grounded to serve as a counter electrode.

In embodiments, the electrostatic charge may be provided using a number of electrodes arranged on both sides of the substrate material 1. The additive 1 may be previously distributed onto the substrate 1. A single-pole output is used to connect biased electrodes to a high-voltage electrostatic generator, while the electrodes located on the opposite side of the substrate 1 are grounded and serve as a counter-electrode.

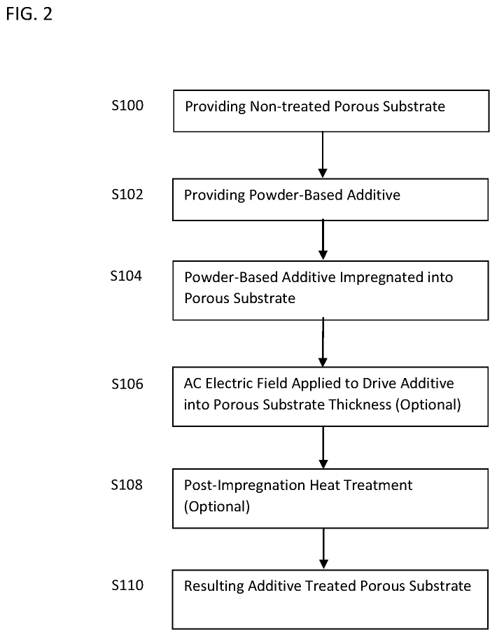

In embodiments, after the additive 2 is added to the substrate 1, a post-impregnation/coating process may improve the adhesion and/or distribution of powder-based additive 2 to substrate 1. In embodiments, as noted above, an AC electric field may be applied to drive the particles of additive 2 into the substrate and/or to provide a desired distribution of the particles throughout the thickness of the substrate. In embodiments, the substrate 1 may be heated to melt the powder-based additive 2 and/or an adhesive/binder system, added to substrate 1 or powder-based additive 2 prior to application, as suggested above, to improve adhesion/bonding of powder-based additive 2 to substrate 1. Additionally, this annealing step may be used produce a uniform layer of the additive 2 covering the exposed surface area of the substrate 1, creating a core-shell coating 2A (see FIG. 2B, for example) which may be used to alter the characteristics or properties exhibited by substrate 1, as discussed above. This heating process may be implemented via a heating element 6, shown in detail in FIG. 1B, for example, through which the substrate 1 passes after the additive is added via the roller 4. After heating, the impregnated substrate 7 including the new characteristics provided by the additive 2 may be removed from the system 100.

In embodiments, the temperature used during the heating process may depend on the purpose of the treatment and materials involved. In embodiments, for treatment of a substrate 1 with powder-based additives 2, the temperature used during the process may dependent on the melt point or activation temperature of the materials used in the adhesive or binder system as well as the tolerances of the additive 2 and substrate material of the substrate 1. In embodiments, the heating process may use a temperature range of 50°-200° C. In embodiments, a heat process may be used to drive off solvents when applying a liquid/gel based additives. In embodiments, the temperature used for this process may be dependent on the solvent and may be less than 120° C.

FIG. 2 illustrates an exemplary flow chart illustrating the steps that may be used to integrated an additive 2 into a substrate 1 using the system 10 discussed above. In embodiments, in step S100, a substrate 1 may be provided to the system 10, for example. As noted above, the substrate may be positioned on a conveyor system, including spindles S1 and S2, for example, and pass through the system 10. At step S102, the additive may be provided to the system. As noted above, in embodiments, the additive 2 may be loaded into the distributor element 3. In step S104, the additive is applied to the substrate. As noted above, in embodiments, the additive 2 may be dropped onto the roller 4 and the roller may contact and integrate or impregnate the additive into the substrate 1 as the substrate passes under the roller 4 as shown in FIG. 1A. In embodiments, in step S106, an AC electric field may be applied across the substrate 1 to distribute the additive 2 across and/or into the substrate. In embodiments, two or more electrodes may be used to apply the AC electric field and the roller 4 may be or may include an electrode. In embodiments, the step S106 is optional. In embodiments, at step S108 the substrate 1, including the impregnated additive 2, may be heat treated via the heating element 6, for example, to improve adhesion of the additive 2 to the substrate 1, to activate one or more characteristics of the additive and/or to drive solvent out of the additive. In step S110, the impregnated and treated substrate 7, which includes the integrated additive may be removed from the system 10, for example via the spindle S2.

The method and system of the present disclosure provide many advantages including: (1) development of cost-effective materials that exhibit characteristics beneficial for the effective treatment of wounds and (2) modulating/altering material characteristics where they are most important (i.e. wound contacting surface).

Although the present invention has been described in relation to particular embodiments thereof, many other variations and modifications and other uses will become apparent to those skilled in the art. It is preferred, therefore, that the present invention be limited not by the specific disclosure herein.

What is claimed is:

1. A system integrating an additive to a substrate comprises:
a distribution element configured to hold a first amount of the additive;
an applicator operably connected to the distribution element to receive the additive and apply the additive to the substrate; and
a conveyor operable to advance the substrate under the applicator,
wherein the additive is applied to the substrate by the applicator such that the additive is integrated into the substrate.

2. The system of claim 1, wherein the additive is a powder.

3. The system of claim 2, wherein the applicator is a roller positioned to contact the substrate such that the roller presses the additive into the substrate.

4. The system of claim 3, wherein the additive includes an adhesive such that the additive remains integrated into the substrate.

5. The system of claim 3, wherein the substrate includes an adhesive configured to retain the additive in the substrate.

6. The system of claim 2, wherein the additive is electrostatically charged.

7. The system of claim 6, further comprising:
a first electrode positioned above the substrate; and
a second electrode positioned below the substrate;
wherein an AC electrical field is established across the substrate between the first electrode and the second electrode to drive the additive through the substrate.

8. The system of claim 7, wherein the first electrode is provided on the roller.

9. The system of claim 7, wherein the first electrode is the roller.

10. The system of claim 7, wherein a distribution of the additive through the substrate depends on the AC voltage field applied between the first electrode and the second electrode.

11. The system of claim 1, wherein the additive is one of a liquid and a semi-liquid.

12. The system of claim 11, wherein the applicator is positioned above the substrate with a gap provided between the applicator and the substrate to allow a layer of the additive to be provided on the substrate.

13. The system of claim 1, further comprising a heating element configured to receive the substrate after the additive is integrated into the substrate to heat the substrate and the additive, wherein the additive at least partially melts to improve adhesion to the substrate.

14. The system of claim 13, wherein the heating element heats the additive to activate one or more characteristics thereof.

15. A method of integrating an additive to a substrate comprising:
providing the additive to a distribution element;
advancing the substrate under an applicator operably connected to the distribution element; and
applying the additive to the substrate as the substrate passes under the applicator such that the additive is integrated into the substrate.

16. The method of claim 15, wherein the additive is a powder.

17. The method of claim 16, further comprising charging the powdered additive electrostatically.

18. The method of claim 17, wherein the step of applying the additive includes applying an AC voltage across a depth of the substrate, wherein varying the AC voltage varies a distribution of the additive through the depth of the substrate.

19. The method of claim 18, further comprising, after the step of applying the additive, applying heat to the substrate and the additive to at least partially melt the additive and improve adhesion to the substrate.

20. The method of claim 19, wherein the step of applying heat to the substrate and the additive activates a characteristic of the additive.

21. The method of claim 15, wherein the additive is one of a liquid and a semi-liquid.

22. The method of claim 21, wherein the step of applying the additive includes applying a layer of the additive on the substrate.
